(19) United States
(12) Patent Application Publication
(10) Pub. No.: US 2016/0089474 A1
(43) Pub. Date: Mar. 31, 2016

(54) Collagen Sponge

(71) Applicants: OSAKA UNIVERSITY, Osaka (JP); KOKEN CO., LTD., Tokyo (JP)

(72) Inventors: Ken NAKATA, Osaka (JP); Yukihiro SATO, Yamagata (JP); Daisuke IKEDA, Tokyo (JP); Ichiro FUJIMOTO, Tokyo (JP)

(21) Appl. No.: 14/767,601

(22) PCT Filed: Feb. 14, 2014
(86) PCT No.: PCT/JP2014/053462

§ 371 Date: Nov. 10, 2015

(30) Foreign Application Priority Data
Feb. 15, 2013 (JP) 2013-027905

(51) Int. Cl. A61L 27/24; A61L 27/56

(52) U.S. Cl. CPC A61L 27/24; A61L 27/56; A61L 2430/06

(57) ABSTRACT

Provided is a collagen sponge which has compressive strength (stress) equivalent to that of a tissue into which the collagen sponge is to be implanted, has no unevenness in structure and stress, and has a pore structure for allowing cells to infiltrate thereinto. The collagen sponge is obtained by subjecting a collagen dispersion, a collagen solution, or a mixture thereof having a collagen concentration of 50 mg/ml or more to freeze-drying and insolubilization treatment thereafter. The collagen sponge thus obtained has a stress of from 10 kPa to 30 kPa when loaded with 10% strain, has in its surface and inside a pore structure having a mean pore diameter ranging from 50 μm to 400 μm, and has a pore diameter standard deviation equal to or less than 80% of the mean pore diameter.

### TECHNICAL FIELD

The present invention relates to a collagen sponge to be used as a substrate for cell culture, implantation into a tissue defect, or the like.

This application claims priority from Japanese Patent Application No. 2013-027905, which is herein incorporated by reference.

### BACKGROUND ART

### Meniscus

A meniscus of a knee joint is a fibrocartilage tissue lying between a femur in the joint and a tibia of a lower leg, and is a tissue to be subjected to various mechanical loads. The meniscus plays a role in shock absorption, load distribution, improvement in ability to slide, stabilization of the joint, or the like. The meniscus is often injured in sports or damaged by daily life activities, and the damage causes a pain of the knee joint and motion limitations. However, the damage is hardly healed naturally.

Hitherto, damaged meniscus which cannot be healed by conservative therapy, such as drug therapy or exercise therapy, has been treated by surgery, and excision or partial excision of the meniscus is performed. Although, such surgery can reduce pain temporarily, functions of the meniscus remain impaired, resulting in arthrosis deformans. In recent years, with an improvement in endoscopic technology, functional preservation has been performed by suture of the meniscus under the sight of an arthroscope.

However, such suture has a problem in that the suture is not applicable to damage with a deficient, complex damage, degenerative tear, and the like, and hence functions of the meniscus cannot be repaired. Such problem occurs not only in the meniscus but also in all cartilage tissues being hardly healed naturally owing to a small number of blood streams.

(Collagen)

As a solution to the problem described above, regenerative medicine has been investigated actively. For example, in Patent Literature 1, there is disclosed “a method involving culturing cartilage cells on a substrate (collagen sponge) for cell culture and implanting the cultured cells into a defect together with the substrate.”

However, the method involves inoculating the cells into the substrate, culturing the cells in vitro for a certain period, and implanting the substrate into a tissue defect, and hence has a problem in that the method can be performed only in a facility with advanced equipment, such as a cell processing center. The method also has a problem of having a difficulty in ensuring safety.

In Patent Literature 2, there is disclosed “a collagen substrate to be used as a graft.” However, the substrate does not have a pore structure in its surface and inside, which inhibits cell infiltration into the inside. The substrate has a small surface area to be brought into contact with a surrounding tissue and a small binding force to a tissue, and hence its bonding to an implantation site is difficult and long-term fixation with a suture thread or the like is required in order to prevent dropping thereof.

Under such circumstances, a patient to whom a substrate derived from collagen has been implanted into a tissue to be subjected to a mechanical load is required to stay in bed until the substrate is bonded to the implantation site. Therefore, a substrate to be implanted into a tissue to be subjected to a mechanical load is required to have a structure easily bonded to the implantation site and to have physical properties equivalent to those of a tissue into which the substrate is to be implanted so that a patient subjected to implantation can resume a daily life immediately.

Further, the substrate is considered to be deteriorated and modified with time in vivo, and hence in order to prevent second surgery for the patient, there is a demand for a substrate which is decomposed in a certain period after implantation into a living body and replaced by a normal autologous tissue.

### CITATION LIST

### Patent Literature

[PTL 1] JP 2008-79548 A
[PTL 2] JP 08-38592 A

### SUMMARY OF INVENTION

### Technical Problem

A substrate, which is derived from collagen and has a pore structure for allowing cells to infiltrate thereinto, is required to have compressive strength (stress) equivalent to that of a tissue into which the substrate is to be implanted and to have no unevenness in structure and stress in order to reduce a mechanical load applied to a tissue around the tissue into which the substrate is to be implanted and maintain the pore structure for allowing cells to infiltrate thereinto. When the stress of the substrate is lower than that of the tissue into which the substrate is to be implanted, the substrate is compressed by the surrounding tissue of the implantation site to cause crush of the pore structure for allowing cells to infiltrate thereinto. Besides, when the stress of the substrate is higher than that of the tissue into which the substrate is to be implanted, the surrounding tissue is provided with physical stimuli to cause inflammation or the like.

It should be noted that the scaffold for cell culture disclosed in Patent Literature 1 using collagen as a raw material has a non-uniform pore structure in which large pores and small pores locally gather (see FIG. 3). The unevenness in the pore structure is highly likely to cause unevenness in stress and partially degrade performance required for the scaffold during use.

### Solution to Problem

The inventors of the present invention have made investigations to solve the problems described above, and have found out that “a collagen sponge, which is obtained by subjecting a collagen dispersion, a collagen solution, or a mixture thereof having a collagen concentration of 50 mg/ml or more to freeze-drying and insolubilization treatment thereafter, and which has a stress of from 10 kPa to 30 kPa when loaded with 10% strain, has in its surface and inside a pore structure having a mean pore diameter ranging from 50 μm to 400 μm, and has a pore diameter standard deviation equal to or less than 80% of the mean pore diameter” has compressive strength (stress) equivalent to that of a tissue into which the collagen sponge is to be implanted, has no unevenness in structure and stress, and has a pore structure for allowing cells to infiltrate thereinto. Thus, the present invention has been completed.

The present invention provides the following items.

1. A collagen sponge, which is obtained by subjecting a collagen dispersion, a collagen solution, or a mixture thereof having a collagen concentration of 50 mg/ml or more to freeze-drying and insolubilization treatment thereafter,

in which the collagen sponge has a stress of from 10 kPa to 30 kPa when loaded with 10% strain, has in its surface and inside a pore structure having a mean pore diameter ranging from 50 μm to 400 μm, and has a pore diameter standard deviation equal to or less than 80% of the mean pore diameter.

2. A collagen sponge according to the above-mentioned item 1, in which the collagen sponge is obtained by performing a centrifugation step at 700 G or more prior to the freeze-drying.

3. A collagen sponge according to the above-mentioned item 1 or 2, in which the pore diameter standard deviation is equal to or less than 60% of the mean pore diameter.

4. A collagen sponge according to any one of the above-mentioned items 1 to 3, in which the pore diameter standard deviation is equal to or less than 40% of the mean pore diameter.

### Advantageous Effects of Invention

The collagen sponge of the present invention is a substrate suited for cell culture under a load, or for implantation into the cartilage tissue defect which has to bear the load.

### BRIEF DESCRIPTION OF DRAWINGS

FIG. 1 is a sectional view (stereoscopic microscope image) of a collagen sponge of the present invention.

FIG. 2 is a sectional view (scanning electron microscope image) of the collagen sponge of the present invention.

FIG. 3 is a sectional view (scanning electron microscope image) of a related-art collagen sponge.

### DESCRIPTION OF EMBODIMENTS

### Collagen Sponge of the Present Invention

A collagen sponge of the present invention has a feature of “being obtained by subjecting a collagen dispersion, a collagen solution, or a mixture thereof having a collagen concentration of 50 mg/ml or more to freeze-drying and insolubilization treatment thereafter, having a stress of from 10 kPa to 30 kPa when loaded with 10% strain, having in its surface and inside a pore structure having a mean pore diameter ranging from 50 μm to 400 μm, and having a pore diameter standard deviation equal to or less than 80% of the mean pore diameter.”

(Collagen to be Used in the Present Invention)

As collagen to be used in the present invention, there may be used: insoluble collagen collected from a tissue in vivo, such as tendon collagen derived from the Achilles tendon or collagen derived from the skin; or soluble collagen or solubilized collagen, such as enzyme-solubilized collagen (atelocollagen), alkali-solubilized collagen, acid-soluble collagen, or salt-soluble collagen, and in particular, the atelocollagen is preferred. The species of animal is not particularly limited, and any collagen having such a denaturation temperature that the collagen does not denature by heat during culture may be used without problems. Specifically, there may be used collagen derived from a mammal, such as cow or pig, collagen derived from a bird, such as chicken, or collagen derived from fish, such as tuna or tilapia. Recombinant collagen may also be used. A side chain of a constituent amino acid of the collagen may be subjected to chemical modification, specifically, acylation, such as acetylation, succinylation, or phthalation, or esterification, such as methylation or ethylation.

(Preparation of Collagen Dispersion, Collagen Solution, or Mixture Thereof)

Prior to a freeze-drying step described later, a collagen dispersion, a collagen solution, or a mixture thereof is prepared. More specifically, insoluble collagen is used as a dispersion, and soluble collagen is used as a solution or a dispersion. The pH of the dispersion, solution, or mixture thereof is not particularly limited, but is preferably approximately neutral, specifically preferably from 4 to 10. It should be noted that the “dispersion” refers to one in a state in which collagen is dispersed or precipitated/swollen without being dissolved at a pH less than pH 1 and more than pH 3 in which the collagen is easily dissolved.

(Adjustment of Collagen Concentration)

In order to achieve strength which is closer to that of the cartilage by freeze-drying, it is necessary to increase, by press, the density of a dried collagen product obtained after the freeze-drying. In the case of increasing the density, pores formed by the freeze-drying are crushed, and hence it is difficult to inoculate cells into the inside of a scaffold. Therefore, it is necessary to adjust the collagen concentration in a dispersion, a solution, or a mixture thereof of insoluble or soluble collagen serving as a raw material for the freeze-drying to 50 mg/ml or more, preferably 70 mg/ml or more, more preferably 100 mg/ml or more. When the collagen concentration is 50 mg/ml or more, a scaffold having physical properties similar to those of a cartilage tissue can be obtained. In particular, when atelocollagen is used, the collagen concentration is preferably 70 mg/ml or more.

It should be noted that when the collagen concentration is low, for example, 30 mg/ml, the physical properties significantly differ from those of the cartilage in vivo. Consequently, it is difficult to transplant the scaffold immediately after inoculation of cartilage cells into the scaffold, to transplant the scaffold after culture, and to culture the inoculated cells while bearing a load to be applied to cartilage cells and a cartilage tissue in vivo. In addition, when the collagen concentration is as low as 30 mg/ml, the physical properties of the collagen sponge of the present invention cannot be obtained even by subsequent insolubilization treatment.

Any one of the dispersion, solution, or mixture thereof having a collagen concentration adjusted as above may be used, but the dispersion is particularly preferred.

(Filling Step)

A mold having a preferred shape is filled with the collagen dispersion, collagen solution, or mixture thereof. The preferred shape may be obtained by making a cubical product and using the product after cutting into the preferred shape before use or by using a mold already having the preferred shape.

A method involving using a mold already having the preferred shape is not particularly limited, but in transplantation into a cartilage defect, the collagen sponge itself is preferably made so as to suit the shape of the cartilage defect.

As a specific method, a mold having a shape corresponding to the shape of a defect may be made by optical modeling based on CT or MRI data of a patient himself.

(Centrifugation Step)

The collagen dispersion, collagen solution, or mixture thereof is preferably subjected to centrifugation before or after the filling step prior to the freeze-drying at preferably 700 G or more, more preferably 750 G or more for preferably from 10 minutes to 200 minutes, more preferably from 15 minutes to 100 minutes. The centrifugation can uniformize the structure and stress of the collagen sponge.

It should be noted that, as shown in Example 1 below, a collagen sponge which has not been subjected to the centrifugation step at 700 G or more has a non-uniform pore structure in the surface and has unevenness.

(Freeze-Drying Step)

As a method for the freeze-drying, a freeze-drying technology which itself is known may be used. Examples of the method for the freeze-drying include rapid freezing and slow freezing. The pore size of a dried product varies depending on the freezing method, and hence a freezing method enabling a preferred pore size is selected. For example, the pore size becomes smaller by rapid freeze-drying and becomes larger by slow freeze-drying.

The pore size is preferably adjusted so that cells can infiltrate into the inside of the collagen sponge and bonding of the cells is not inhibited by a body fluid flowing into and out of the collagen sponge. Therefore, the pore size is preferably adjusted so as to achieve a mean pore diameter of from 50 μm to 400 μm. The preferred pore size may be achieved by leaving a container filled with the collagen dispersion to standstill on a shelf cooled to −20° C. in a freeze-drying machine to freeze the dispersion and then drying the resultant under reduced pressure for from 70 hours to 75 hours with increasing the temperature over time from −20° C. to normal temperature.

(Insolubilization Treatment Step)

Insolubilization treatment can increase physical strength and adjust a remaining period in a tissue into which the sponge collagen has been transplanted. The insolubilization treatment needs to be performed uniformly into an inside of the dried collagen product without deforming a dried collagen product.

The insolubilization treatment in the present invention is preferably dry-heat treatment, γ-ray irradiation treatment, treatment using a water-soluble chemical cross-linking agent, or treatment using a vaporable chemical cross-linking agent, which enables insolubilization treatment into the inside of the dried product.

The insolubilization treatment is differently performed depending on a method employed. For example, the dry-heat treatment may be performed by achieving a completely dried state and leaving the dried product to stand for 30 minutes or more under a heating atmosphere of about 120° C., and the γ-ray irradiation treatment may be performed by giving moisture to the dried product to the extent that the product is not swollen and performing irradiation at 0.1 kGy or more. In the insolubilization treatment using a water-soluble chemical cross-linking agent, specifically, an aldehyde compound, an epoxy compound, or the like may be used. For example, when glutaraldehyde is used, the insolubilization treatment may be accomplished by immersing the dried product in an aqueous solution containing glutaraldehyde at a concentration of 0.5%.

In the insolubilization treatment using a vaporable chemical cross-linking agent, the insolubilization treatment is performed in a sealed container by adding the dried product and a chemical cross-linking agent, such as a formalin solution, to the sealed container.

As the water-soluble chemical cross-linking agent to be used in the present invention, a chemical cross-linking agent as an epoxy compound is preferably used, and the following chemical cross-linking agent is more preferably used: allyl glycidyl ether, 2-ethylhexyl glycidyl ether, phenyl glycidyl ether, phenol (EO) 5 glycidyl ether, p-tert-butylphenyl glycidyl ether, dibromophenyl glycidyl ether, lauryl alcohol (EO) 15 glycidyl ether, resorcinol diglycidyl ether, neopentyl glycol diglycidyl ether, 1,6-hexanediol diglycidyl ether, glycerin polyglycidyl ether, trimethylolpropane triglycidylether, pentaerythritol polyglycidyl ether, diglycerin polyglycidyl ether, polyglycerin polyglycidyl ether, sorbitol polyglycidyl ether, diglycidyl terephthalate, ethylene glycol diglycidyl ether, diethylene glycol diglycidyl ether, polyethylene glycol diglycidyl ether, polybutadiene diglycidyl ether, or the like.

In the present invention, the insolubilization treatment is performed so that the stress of a substrate is equivalent to that of a cartilage tissue. In daily life, the cartilage tissue has a deformation ratio (strain) of about 10% when compressed in a vertical direction and has a stress under the condition of from 10 kPa to 30 kPa. Therefore, the conditions of the insolubilization treatment are adjusted so that the stress of the substrate of the present invention is also from 10 kPa to 30 kPa when loaded with 10% strain. Further, the insolubilization treatment also provides an effect of preventing early decomposition and absorption of the substrate in vivo.

(Application of Collagen Sponge of the Present Invention)

The collagen sponge of the present invention may be implanted without additional treatment or may be implanted after cells are inoculated into the collagen sponge and cultured prior to the implantation. In the culture, the cells may be cultured while a load similar to that to be applied to a cartilage tissue in vivo is applied thereto.

Further, the collagen sponge of the present invention may be implanted into a living body as a drug scaffold or a reinforcement for a tissue other than the cartilage or may be used as a scaffold for cell culture not intended to be implanted into a living body. In particular, the collagen sponge of the present invention may also be used as a scaffold for cell culture under repetitive loads.

(Characteristics of Collagen Sponge of the Present Invention)

The collagen sponge of the present invention has a stress of from 10 kPa to 30 kPa, specifically from 15 kPa to 25 kPa when loaded with 10% strain. It should be noted that the stress when loaded with 10% strain is measured for the collagen sponge of the present invention immersed in physiological saline at 37° C. using a small desktop testing machine (Shimadzu EZ-S).

The mean pore diameter of the collagen sponge of the present invention ranges from 50 μm to 400 μm, specifically from 60 μm to 300 μm, more specifically from 70 μm to 200 μm.

In addition, the amount of variation of the pore diameters of the collagen sponge of the present invention, i.e., the standard deviation is equal to or less than 80%, preferably equal to or less than 60%, more preferably equal to or less than 40% of the mean pore diameter. A value of “the pore diameter standard deviation is equal to or less than X % of the mean pore diameter” in the present invention refers to a value calculated by dividing a value of the pore diameter standard deviation by the mean pore diameter (value of pore diameter standard deviation/mean pore diameter).

On the other hand, the pore diameter standard deviation of a related-art collagen sponge is about 84% (=standard deviation: 96.40 μm/mean diameter: 114.88 μm) of its mean pore diameter.

The present invention is described by way of Examples below. However, the present invention is by no means limited to these Examples.

### Example 1

### Production of Collagen Sponge of the Present Invention

A collagen sponge of the present invention was produced by the following steps.

(Production Method for Collagen Sponge of the Present Invention)

Enzyme-solubilized collagen (atelocollagen) derived from bovine skin was added to purified water, and the resultant was stirred by a kneader, thereby yielding a collagen dispersion. The collagen concentration in the dispersion was measured by a burette method, and the collagen concentration in the dispersion was adjusted to 100 mg/ml. It should be noted that, when the concentration was lower than 100 mg/ml, the enzyme-solubilized collagen was added to increase the concentration.

The collagen dispersion having a collagen concentration of 100 mg/ml was dispensed into a Teflon (trademark) tube and centrifuged at 760 G for 20 minutes. After the centrifugation, the dispersion was left to stand still on a shelf cooled to −20° C. in a freeze-drying machine to be frozen, and the resultant was dried under reduced pressure for 73 hours with increasing the temperature over time from −20° C. to normal temperature.

After completion of the freeze-drying, the dried collagen product was taken out from the Teflon (trademark) tube, cut into a required length. The dried collagen product was added to a media bottle containing an epoxy compound serving as a cross-linking agent, and the mixture was deaerated under reduced pressure (5 minutes×three times) and stirred by shaking (50 rpm, 30° C., 18 hours) to perform insolubilization treatment.

Subsequently, the collagen product after the insolubilization treatment was washed by: transferring the collagen product to a media bottle containing ion exchange water; performing stirring by shaking (50 rpm, 30° C., 30 minutes×5 times); and then adding a neutralizing solution for the cross-linking agent; performing stirring by shaking (50 rpm, 30° C., 18 hours); transferring the resultant again to a media bottle containing ion exchange water; and performing stirring by shaking (50 rpm, 30° C., 30 minutes×5 times).

Finally, the resultant was air-dried, thereby yielding a collagen sponge of the present invention.

It should be noted that a related-art collagen sponge obtained without performing the centrifugation at 760 G for 20 minutes was used as a control (see a collagen sponge disclosed in Patent Literature 1).

(Characteristics of Collagen Sponge of the Present Invention)

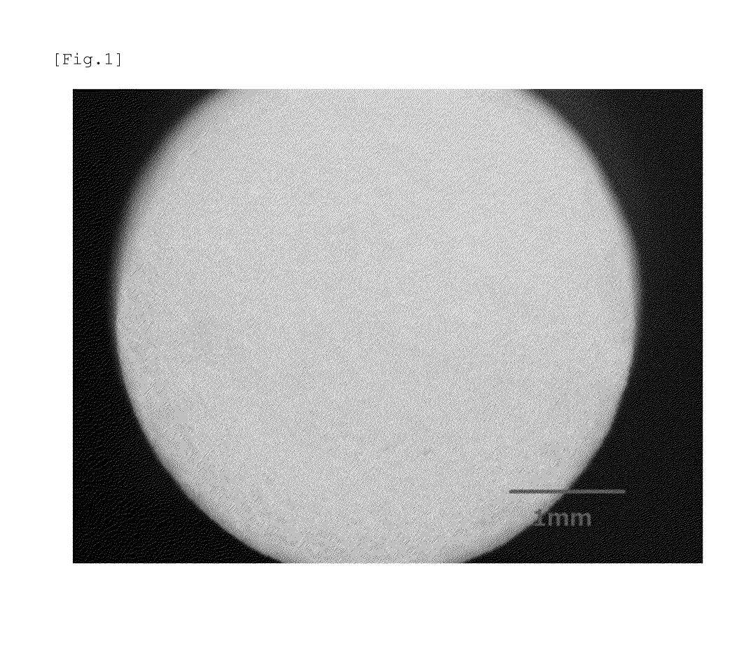

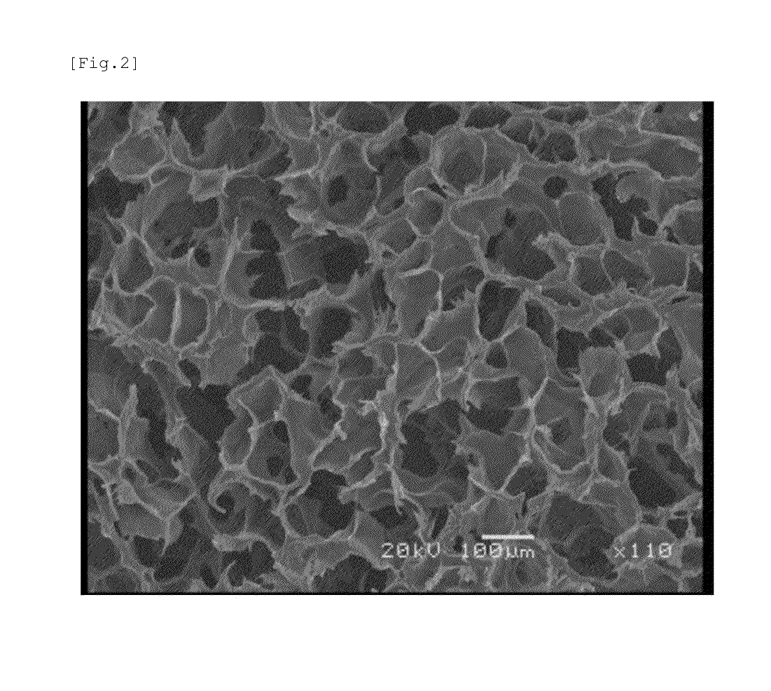

Cross sections of the collagen sponge obtained above are shown in FIG. 1 (stereoscopic microscope image, Bar: 1 mm) and FIG. 2 (scanning electron microscope image, Bar: 100 μm). As is apparent from FIG. 1 and FIG. 2, the collagen sponge of the present invention was found to have, in its surface, a uniform pore structure and have no unevenness.

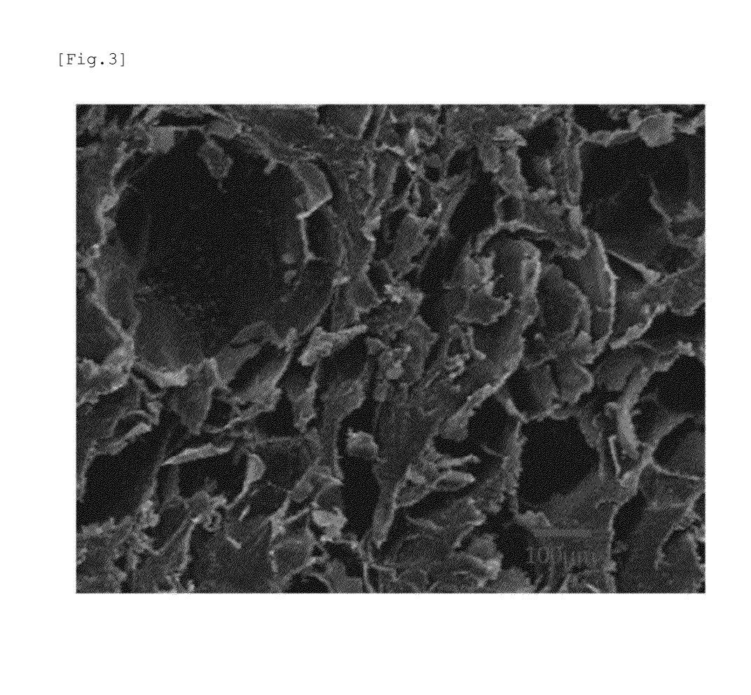

On the other hand, a cross section of the related-art collagen sponge obtained above is shown in FIG. 3 (scanning electron microscope image, Bar: 100 μm). As is apparent from FIG. 3, the related-art collagen sponge was found to have, in its surface, a non-uniform pore structure and have unevenness.

The stresses of the collagen sponge of the present invention and the related-art collagen sponge were measured. By referring to the deformation degree (10% strain) of the meniscus by a load to be applied to the knee in daily life, the stress was measured when the scaffold was compressed with 10% strain. The stress was measured for the collagen sponge of the present invention immersed in physiological saline at 37° C. using a small desktop testing machine (Shimadzu EZ-S).

The collagen sponge of the present invention was found to have a stress of about 18.7 kPa when loaded with 10% strain.

On the other hand, the related-art collagen sponge was found to have unevenness in many parts and have a stress of about 14.0 kPa when loaded with 10% strain in a certain part.

One pore was selected randomly from the surface (scanning electron microscope image) of each of the collagen sponge of the present invention and the related-art collagen sponge, and the long diameter of the pore and the diameter passing through the center of the long diameter in a direction perpendicular to the long diameter were measured, followed by calculation of a mean value of the two values as a pore diameter. The procedure was performed for 25 pores, and the mean pore diameter and pore diameter standard deviation were calculated.

The collagen sponge of the present invention was found to have a mean pore diameter of 115.65 μm and a pore diameter standard deviation of 36.18 μm.

On the other hand, the related-art collagen sponge was found to have a mean pore diameter of 114.88 μm and a pore diameter standard deviation of 96.40 μm.

### Example 2

The collagen sponge produced in Example 1 was implanted into an animal, and implantation evaluation was performed. Details are as described below.

(Implantation Method)

A cylindrical defect with a diameter of 5 mm was made in an anterior segment of the medial meniscus of each of nine miniature pigs, and analyses were performed on three months after the surgery for respective 6 cases of three groups, group A (implanted with the collagen sponge produced in Example 1), group B (negative control: not implanted), and group C (positive control: implanted with fibrin clot).

(Results of Implantation)

In the three groups, infection, adhesion, and arthritis were not observed visually. According to histological evaluation, in the group A implanted with the collagen sponge produced in Example 1, cell invasion, partial absorption, and fibrous tissue replacement were observed, and the surrounding tissue were maintained well as compared to the case of the group B.

Tissue filling ratios of the group A, the group B, and the group C were 91%, 52%, and 68%, respectively. That is, the implantation with the collagen sponge produced in Example 1 is provided with a significantly high tissue filling ratio as compared to the control implantation.

Regarding tissue scoring (Ishida K et al, Tissue Eng, 2007), there was no significant difference among the three groups.

The evaluation for the meniscus-deficient pig models suggested that the collagen sponge of the present invention was excellent in tissue induction and maintenance of the surrounding tissue as compared to the fibrin clot. Further, transplantation of the collagen sponge of the present invention into a tissue in vivo caused no adverse phenomena, such as infection, tissue adhesion, and arthritis.

### INDUSTRIAL APPLICABILITY

The collagen sponge, being the substrate suited for the cell culture while applying the load, or for the implantation into the cartilage tissue defect while bearing the load, could be provided.

1. A collagen sponge, which is obtained by subjecting a collagen dispersion, a collagen solution, or a mixture thereof having a collagen concentration of 50 mg/ml or more to freeze-drying and insolubilization treatment thereafter,
wherein the collagen sponge has a stress of from 10 kPa to 30 kPa when loaded with 10% strain, has in its surface and inside a pore structure having a mean pore diameter ranging from 50 μm to 400 μm, and has a pore diameter standard deviation equal to or less than 80% of the mean pore diameter.

2. A collagen sponge according to claim 1, wherein the collagen sponge is obtained by performing a centrifugation step at 700 G or more prior to the freeze-drying.

3. A collagen sponge according to claim 1, wherein the pore diameter standard deviation is equal to or less than 60% of the mean pore diameter.

4. A collagen sponge according to claim 1, wherein the pore diameter standard deviation is equal to or less than 40% of the mean pore diameter.
